SEQUENCE LISTING (2 sequences)

SEQ ID NO: 1 - DNA, 1301 bp, Artificial Sequence
  Fusion of a splice acceptor, linker, PTV1-2A peptide, mus musculus CD79A open reading frame, mus musculus CD79A untranslated region, and polydenylation sequence
cagggatccggagccacgaacttctctctgttaaagcaagcaggagacgtggaagaaaaccccggtcctctgcgggtagaagggggtccaccatccctgacggtgaacttgggcgaggaggcccgcctcacctgtgaaaacaatggcaggaaccctaatatcacatggtggttcagccttcagtctaacatcacatggcccccagtgccactgggtcctggccagggtaccacaggccagctgttcttccccgaagtaaacaagaaccacaggggcttgtactggtgccaagtgatagaaaacaacatattaaaacgctcctgtggtacttacctccgcgtgcgcaatccagtccctaggcccttcctggacatgggggaaggtaccaagaaccgcatcatcacagcagaagggatcatcttgctgttctgtgcagtggtgccagggacgctgctgctattcaggaaacggtggcaaaatgagaagtttggggtggacatgccagatgactatgaagatgaaaatctctatgagggcctgaaccttgatgactgttctatgtatgaggacatctccaggggactccagggcacctaccaggatgtgggcaacctccacattggagatgcccagctggaaaagccatgactgacatgtcccacccttccctgcctgccatatgtctgactccagcatccctgcctccccactcttcccctggagtccccccttcccagtgcttgctccagccactcgagtggcctacccttgcctccaggctgccccaccctattctcccacctaatcttgtccttccccaagtccagctccctctctagtgggtaatgagcccttaatcgctgcctctaggggagctgattacagcagcctcgttagtgtcaccccccctcctcccagatctgtcatggccacttaagtgataataaatccttcccaaagcagcccttggcaggagtcatggatctcatgtagactgctcctcaacactgccagggaaccaggagcagaaaacccaagtcccctcccccctcagacccaggattctaggttcaagttctcacctgaccacttctgagatctgggaccagacagttcttgctgtctgtcttccccaaccccctccccccaaaataacaggaagtctcttggggaagtccccaccccaattgaaatgctggtttatctcccttatagggcctcaccctcctcatgtcccaaagcaagggggaaccagcccgggtgttaccaaatttttacctatatttaaactctgagaacac

SEQ ID NO: 2 - DNA, 1950 bp, Artificial Sequence
  Fusion construct of splice acceptor, linker, PTV1-2A peptide, mus musculus Tnfrsf13c (tumor necrosis factor receptor superfamily member 13c) open reading frame, mus musculus Tnfrsf13c untranslated region and polyadenylation sequence
cagggatccggagccacgaacttctctctgttaaagcaagcaggagacgtggaagaaaaccccggtcctatgggcgccaggagactccgggtccgaagccagaggagccgggacagctcggtgcccacccagtgcaatcagaccgagtgcttcgaccctctggtgagaaactgcgtgtcctgtgagctcttccacacgccggacactggacatacaagcagcctggagcctgggacagctctgcagcctcaggagggctccgcgctgagacccgacgtggcgctgctcgtcggtgcccccgcactcctgggactgatactggcgctgaccctggtgggtctagtgagtctggtgagctggaggtggcgtcaacagctcaggacggcctccccagacacttcagaaggagtccagcaagagtccctggaaaatgtctttgtaccctcctcagaaacccctcatgcctcagctcctacctggcctccgctcaaagaagatgcagacagcgccctgccacgccacagcgtcccggtgcccgccacagaactgggctccaccgagctggtgaccaccaagacagctggcccagagcaatagcagcagtggaggctggaacccagggatctctactgggcttgtggacttcacccaacagcttgggaaagaacttggcccttcagtgacggagtcctttgcctggggggcgaacccggcagaaccagacactacaggccacatgagattgcttttgtgttagctcttgacttgagaacgttccatttctgagatggtttttaagcctgtgtgccttcagatggttggatagacttgagggttgcatatttaatctctgtagtgagtcggagactggaaacttaatctcgttctaaaaattttggattactgggctggaggtatggctcagcagttcggtttgtgtgctgttctagccgaggactccagttgttcagcttcccggaactcagatctggcagcttaagaccacctgtcactccagcccctggaacatccttgcctccaaaggcaccagcactcatttgctctagagcacacacacacacacacacacacacacacacacacacacacacatatgcatgcatgcacacttaaaaatgtcaaaattagcggctggagaaattcatggtcaacagcgcttactgtgattccagaggatgagagtttgattcccagaatgcactgcgggtggctcattactgagcataacttttgcttcaggggacctgatgcctctggacttcatgggcatctgtattcacgtgcacatcctacacacacacacacacacacacacagacatacacacacacacactcttttacaaatgataaaatataagatgggcatggtggtacacacctttaatcccaacattggggaagcaaaggcaggcaggtaactgagttggaggccatcctggtctacatagcaagttccaggctaaccagagctaaatggtgagaccaagtctcaaaataatactcccccccccaaaaaaaaaacttttaaattttgatttttttcttttattattattttttatattaatttcatggtgtttagaagtggtatacttagatggtgactaagaggaggtaaagccatcaggactgagcccctaacatacaaggagaaagcagagacaatgaacacgcccctctcctgctgtgtgccagctctggaccaccagccagagggcaatcatcagatgtgggccctagaaccttcagagccgaaagctaaatcaatctcatttctttgtaaagctatttagccttaggtgttttgttacggtgatataaaatggactaacacaggcactatgagtaagaagcttttctttgagctgggaaaggtactgttaaaccaaaattaatctgaataaaaaaaggctaaggggaagacact

(19) United States
(12) Patent Application Publication
(10) Pub. No.: US 2018/0230238 A1
(43) Pub. Date: Aug. 16, 2018

(54) ENHANCED PRODUCTION OF IMMUNOGLOBULINS

(71) Applicant: Trianni, Inc, San Francisco, CA (US)

(72) Inventors: Matthias Wabl, San Francisco, CA (US); Nigel Killeen, San Francisco, CA (US)

(21) Appl. No.: 15/751,015

(22) PCT Filed: Aug. 24, 2016
(86) PCT No.: PCT/US16/48410

§ 371 Date: Feb. 7, 2018

Related U.S. Application Data
(60) Provisional application No. 62/209,267, filed on Aug. 24, 2015.

(51) Int. Cl. C07K 16/46; A01K 67/027

(52) U.S. Cl. CPC C07K 16/468; A01K 67/0275; A01K 2217/07; A01K 2217/072; A01K 2227/105; C07K 2317/20; C07K 2317/31; A01K 2267/01

(57) ABSTRACT

The present invention provides cells, transgenic animals, including transgenic mammals and particularly rodents, comprising engineered immunoglobulin alleles. Mutations in the alleles are designed to compromise allelic exclusion and have potential to be exploited for the isolation of bispecific antibodies.

### RELATED APPLICATIONS

This application is a National Phase filing under 35 U.S.C. 371 of International Patent Application No. PCT/US2016/048410, filed Aug. 24, 2016, which claims the benefit of priority to U.S. Provisional Application No. 62/209,267, filed Aug. 24, 2015 and is incorporated herein by reference.

### FIELD OF THE INVENTION

This invention relates to production of immunoglobulin molecules, including production of bispecific antibodies in transgenic vertebrates.

### BACKGROUND OF THE INVENTION

In the following discussion certain articles and methods are described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

IgG antibodies in most vertebrates exist as dimers of two identical heavy (H) chains; each of the H chains is paired with an identical light (L) chain. A variable domain at the N-terminus of each H and L chain, VH and VL respectively, provides each H/L chain pair with its unique antigen-binding specificity. Normally each IgG antibody consists of two identical antigen-binding sites and is monospecific. However, for certain therapeutic uses, for example in cancer immunotherapy, a bispecific antibody that can bind two different antigens would be advantageous.

The exons encoding the VH and VL domains do not exist in the germ line DNA or in any somatic cell except for the B lymphocyte. During B cell development, the VH exon is formed by the assembly of randomly selected V, D and J gene segments, a process called V(D)J rearrangement. A similar process involving V and J gene segments then occurs to form the VL exon. In humans and most other higher vertebrates, there are two alleles that can potentially express the H chain, two alleles that can express the kappa (κ) L chain, and two alleles that can express the lambda (λ) L chain. The H chain locus contains multiple V, D and J gene segments that can be used to form the VH exon, and each κ and λ L chain locus contains multiple V and J gene segments that can be used to form a VL exon. The H chain locus also contains exons to encode the different antibody classes (isotypes). Despite the presence in the genome of multiple immunoglobulin loci, each with two alleles, individual B cells only expresses one functional H chain and one functional L chain, a process called allelic exclusion, thus guaranteeing monospecificity.

During B cell development, V(D)J rearrangement occurs first at one of the H chain alleles to produce a full length IgM H chain (μ HC). The cell can “sense” a successful V(D)J rearrangement because the newly synthesized μ HC pairs with VpreB and λ5 proteins in the endoplasmic reticulum to form the pre-B cell receptor (pre-BCR) complex. The pre-BCR traffics to the cell surface and, by incompletely understood mechanisms, signals to terminate the expression of enzymes essential for V(D)J rearrangement; thus, only one H chain allele is rearranged. However, the DNA recombination process is inherently error prone, so that many of the V(D)J rearrangements on the first H chain allele are non-productive, i.e., incapable of producing the H chain protein. In this situation, there is no pre-BCR formed, therefore V(D)J rearrangement can occur on the second H chain allele.

After successful V(D)J rearrangement and production of a μ HC, VJ rearrangement commences and continues until a LC protein is produced. The process starts at one of the κ L chain alleles and, if necessary, moves to the λ locus. After successful VJ rearrangement, the LC produced pairs with the HC homodimer to form the B cell receptor, which then traffics to the plasma membrane where it signals to terminate any further L chain gene rearrangement. A common feature of both H chain and L chain allelic exclusion is the expression of a HC on the cell surface, as a component of either the pre-BCR or BCR.

Because of allelic exclusion at the H and L chain loci, each B lymphocyte is capable of producing only monospecific antibodies. Therapeutically, however, artificially engineered antibodies that harbor two different antigen-binding sites per antibody molecule have proven to be efficacious as treatments for a number of diseases. The generation of such bispecific antibodies typically involves time-consuming separate efforts to screen, identify, and isolate the monospecific antibodies against each the two distinct antigens of interest. Subsequently, the genes encoding the H and L chains of each candidate monoclonal antibody to be engineered as one half of a bispecific antibody are cloned and modified for permissive heterotypic associations between H chains or between H and L chains. To obtain bispecific antibodies for further evaluation, a cell line must be transfected with the modified H and/or L chain genes from the two original monoclonal antibodies. Thus, a method for more efficient production of bispecific antibodies, particularly during the initial phases of drug development, is an important unmet need. The present invention addresses this unmet need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides facilitated processes for the isolation of bispecific antibodies, comprising transgenic animals, including transgenic mammals, carrying modified immunoglobulin loci or other transgenes in their genomes. The modifications to the loci interfere with the normal mechanisms of allelic exclusion following VDJ and/or VJ rearrangements. By interfering with the signals that mediate allelic exclusion during B cell development, the invention described herein provides methods for the rapid isolation of bispecific antibodies from vertebrate animals including humans and rodents. Modifying the genomic contents of animals so that their B cells are capable of expressing more than one functional V_{H} domain paired with one or more functional V_{L} domain per cell provides for much more efficient production of bispecific antibodies. Suppressing allelic exclusion to facilitate the production of bispecific antibodies from an immunized host is a significant step forward in such production.

In certain embodiments, the invention introduces modifications to the immunoglobulin alleles to impinge on the normal allelic exclusion processes such that an in-frame V(D)J rearrangement on one chromosome does not inhibit V(D)J rearrangement on the other homologous chromosome. Methods for generating the modifications and how they could be exploited to facilitate the isolation of bispecific antibodies are set forth in more detail herein.

In another embodiment, the invention provides modifications to immunoglobulin alleles that impinge on the normal allelic exclusion process such that V(D)J rearrangement on both homologous chromosomes in individual developing B cells is favored relative to the normal situation. The relevant modifications interfere with the expression of full-length immunoglobulin chains from one of the two chromosomes, but do so in a fashion that can be reversed in an inducible fashion.

Compromised allelic exclusion is associated with “allelic inclusion”, which can be defined as an atypically increased frequency of B cells carrying rearranged heavy chain genes on both homologous autosomes. B cells with productively rearranged heavy chain genes on both homologous chromosomes are a potential source of bispecific antibodies because the two rearrangements are expected to have occurred independently and thus will encode heavy chains with different variable domains. The present invention comprises methods for increasing allelic inclusion and thereby creating conditions permissive for the isolation of bispecific antibodies.

An advantage of the current invention is that it provides a means for isolating primary B cells or their hybridoma derivatives that are capable of expressing bispecific antibodies because of compromised allelic exclusion. That is, allelic inclusion confers on individual B cells, or their derivatives, the capacity to express two physically distinct antibody molecules. One of the antibody molecules contains a heavy chain from one of the two rearranged heavy chain genes, while the second antibody contains a heavy chain from the other rearranged gene. Bispecific antibodies can be expressed in such cells by combining the two different heavy chains together into one antibody molecule forming heterodimers (see Example 1).

A result of the invention as just outlined is that light chain diversity is extensive in B cells; that is, normally individual B cells with the capacity to express two different heavy chains would be expected only to express one kind of light chain. Thus the two antibodies such B cells generate each have different heavy chains dimerized with a common light chain. Whereas different bispecific B cells express different light chains, each such B cell would express only one light chain.

An alternative embodiment of the invention is one that involves compromising allelic exclusion at the light chain loci rather than at the heavy chain locus. In this version of the invention, individual B cells have an enhanced likelihood of featuring allelic inclusion at a light chain locus, e.g., the kappa locus, and thus they have an enhanced likelihood of expressing two distinct light chain proteins (See Example 2).

In the light chain allelic inclusion scenario, B cells feature normal heavy chain allelic exclusion, and thus the vast majority of these B cells—like normal B cells—express only one heavy chain protein. Because of allelic inclusion at a light chain locus, however, this heavy chain protein may be dimerized with one of two distinct light chain proteins, creating a situation in which a single B cell produces (in an exclusive, sequential or simultaneous fashion) two different antibody molecules featuring distinct antigen-binding properties.

Compromising allelic exclusion operating on either the heavy chain locus or a light chain locus increases the likelihood that individual B cells will express two distinct antibody molecules. In the heavy chain allelic inclusion scenario, the two antibody molecules share a common light chain but differ in their heavy chains. By contrast, in the light chain allelic inclusion scenario, the two antibody molecules share a common heavy chain but differ in their light chains. For some combinations of antigens one scenario may be better than the other in terms of the ease with which bispecific B cells are isolated. Both, however, represent viable scenarios from which to isolate such cells.

The two scenarios for accomplishing allelic inclusion (heavy chain-centered versus light chain-centered) share a common mechanistic theme. In both cases, one of two homologous immunoglobulin alleles is compromised in its capacity to generate a fully functional B cell antigen receptor molecule. Specifically, the protein product of the compromised allele is not able to participate in normal antibody assembly in cells, and/or it is not able to participate in signal transduction necessary for B cell development and/or B cell antigen responses (i.e., antigen-driven clonal expansion and B cell differentiation).

In some aspects, the genetically modified animal comprises an immunoglobulin heavy or light chain gene, wherein part or all of the constant region exon(s) have been placed in inverted reading frame orientation relative to rearranged V(D)J gene segments in the same allele. The modification renders the constant domain-encoding part inaccessible or otherwise nonfunctional on the same DNA strand as the coding region of the variable region gene segments. The mutation may, in one aspect, be rendered inducible through use of appropriately placed site-specific recombinase sites, which are recombined in an inducible fashion by the relevant recombinase in such a way as to confer functionality where it was previously lacking. As an example, the conferment of functionality may be accomplished by inverting the piece of DNA containing the constant domain-encoding exon so that it is now on the same strand of DNA as that of the variable region gene segments in the same allele.

The inducible aspect of the mutations just outlined is advantageous because it permits the full functionality of the immunoglobulin chain alleles to be turned on or off at specific times. Most importantly, when the mutant alleles are present in a transgenic mouse, the alleles can be turned on or off in this fashion during immunization schemes, and in turn used for the isolation of bispecific antibodies.

V(D)J rearrangement is productive in only one-third of the cases, which reduces the available antibody repertoire. Another major advantage of the present invention is that it provides methods to select for B cells that have productively assembled two V(D)J gene segments per heavy chain or light chain locus. In some embodiments, a DNA cassette is inserted not only to prevent expression of a functional immunoglobulin heavy or light chain, but also to provide the necessary signals for B cell development and/or survival only when an in-frame V(D)J exon has been assembled.

In certain configurations of this aspect, two rearranged immunoglobulin heavy chain genes in individual B cells in the animal express gene products that favor heterodimerization with each other over homodimerization. Yet other configurations provide B cells of the animal, wherein heterodimerization of the two gene products is enabled by a developmental or differentiation event, or can be induced.

The invention thus provides genes for immunoglobulin heavy or light chains—and transgenic animals carrying these genes—that have been engineered such that allelic exclusion of the genes during V(D)J rearrangement is compromised. It further provides mutations in the constant domain-encoding parts of the immunoglobulin heavy or light chain genes or locales that are responsible for causing the allelic inclusion phenotypes. The mutations have an inducible aspect, which permits immunization schemes to be used that would allow for the isolation of bispecific B cells, i.e., B cells with the capacity to express bispecific antibodies. It further provides additional modifications of the heavy chain locus to promote heterodimer formation, while suppressing homodimerization, between the two heavy chains present in the bispecific B cells.

These and other aspects, objects and features of the invention are described in more detail below.

### BRIEF DESCRIPTION OF THE FIGURES

FIG. 1. Heavy chain alleles on two homologous chromosomes in committed lymphoid progenitors in a mouse prior to V(D)J rearrangement. The heavy chain alleles (101 and 102) in this embodiment are comprised of chimeric V, D and J gene segments containing human coding regions and mouse non-coding regions, although other kinds of gene segments are compatible with the invention and can also be employed. In this context, as with other alleles described herein, an “allele” such as 101 and 102 refers to a chromosome segment that may include V, D, and J genes, an intronic enhancer, constant regions genes, and other sequences. Depicted are the V (103), D (104), and J (105) gene segments, which undergo recombination during B cell development, the heavy chain intronic enhancer (106) involved in transcription of the H chain locus, the switch μ (s-μ) region (107) involved in isotype switching to downstream constant region genes (not shown), and the constant (C) domain exons (108), which in a physiological context would encode mu (Cμ) and delta (Cδ). Labeled components in the figure are as follows: Chromosomal segments at the heavy chain locus (101, 102); Variable region gene segments (103); D region gene segments (104); J region gene segments (105); Heavy chain intronic enhancer (106); Switch region (107); Exons encoding the constant regions of IgM and IgD (108).

FIG. 2. Heavy chain alleles found on two homologous chromosomes in committed lymphoid progenitors in a mouse prior to V(D)J rearrangement. In this FIG. 2, the V (203), D (204) and J (205) gene segments are compressed compared to FIG. 1 so as to emphasize the structure of the constant region locale of the locus. The heavy chain enhancer (206) and s-regions (207) correspond to elements 106 and 107 in FIG. 1. The Cμ and Cδ exons (208) are used in production of μ and δ HC proteins. IgM and IgD are co-expressed on mature B cells and have the identical VH region because they are derived from the same primary transcript 5′-VDJ-Cμ-Cδ-3′. During an immune response, the antigen-specific B cell can switch from production of IgM/IgD to production of IgG subclasses, IgE or IgA by a DNA deletion mechanism that replaces the Cμ and Cδ exons (208) with one of the downstream C-regions. In the C57BL/6 mouse these are Cγ3 (209), Cγ1 (210), Cγ2b (211), Cγ2c (212) Cε (213) and Cα (214). Mouse strains such as BALB/c have Cγ2a instead of Cγ2c. There are s-regions (207) upstream of all C-region genes except for Cδ. Labeled components in the figure are as follows: Heavy chain alleles (201, 202); Variable region gene segments denoted in brackets (203); D region gene segments denoted in brackets (204); J region gene segments (205); Heavy chain intronic enhancer (206); Switch region (207); IgM and IgD (208, individual exons not shown); C_{H} exons of IgGs and other antibody classes (209-214, individual exons not shown).

FIG. 3. A combination of heavy chain alleles permissive for isolation of bispecific antibodies featuring a genetic modification to select for allelic inclusion. FIG. 3 depicts the heavy chain alleles found on two homologous chromosomes in committed lymphoid progenitors in a mouse prior to V(D)J rearrangement. The heavy chain alleles (301 and 302) are comprised of chimeric gene segments containing human coding regions and mouse non-coding regions, although other kinds of gene segments are compatible with the invention and thus can be employed. Again, in this context and as with other alleles described herein, an “allele” such as 301 and 302 refers to a chromosome segment that may include V, D, and J genes, an intronic enhancer, constant regions genes, and other sequences, including elements 303 to 317, described below. After V(D)J rearrangement, but prior to appropriate site-specific recombination, allele 301 is not capable of expressing a full-length heavy chain protein because the open reading frame of its constant domain exons (309) is in an antisense orientation relative to the open reading frames of the V gene segments (303) in the same allele. By contrast, allele 302 is capable of expressing a full-length heavy chain protein because the open reading frame of its constant domain exons (310) is in a sense orientation relative to the open reading frames of the V gene segments (303) in the same allele. Allele 301 includes an element (311) that allows for in vivo selection for productive V(D)J rearrangements on this allele; translation of this element only occurs if an upstream rearranged VDJ exon contains a translated unbroken open reading frame, and the protein product of the 311 element is required for completion of B cell development and/or B cell antigen receptor signaling or function. Allele 302 also includes the same sort of element (311), but in an inverted translational orientation relative to the open reading frames in the V gene segments in the same allele; because of this inverted orientation, the element is not translated until after site-specific recombination (involving sites 307 and 308). Allele 302 is capable of undergoing limited switch recombination from an IgM constant domain-encoding set of exons (310) to an IgG constant domain-encoding set of exons (312). Allele 302 includes an additional component (313) that permits identification of cells that have participated in clonal expansion and isotype switching prior to site-specific recombination involving sites 307 and 308. The 313 element is part of the IgG constant domain-encoding set of exons (312) and in one version of the invention it is an IRES-Flp or similar element. Cells that have not undergone induced site-specific recombination at the 302 allele—but have undergone switch recombination during antigen responses—express the Flp protein from the IRES-Flp element. B cells expressing the Flp protein can be marked through use of an appropriate reporter transgene (e.g., Rosa26-FRT-Stop-FRT-YFP). Both alleles (301 and 302) contain elements (316 and 317) that promote heterodimerization of the two different heavy chains produced in the bispecific B cell, hybridoma or other derivative. The heterodimerizers are activated after site-specific recombination and deletion involving the 314 and 315 elements on both alleles. Labeled components in the figure are as follows: Variable region gene segments (303); D region gene segments (304); J region gene segments (305); Heavy chain intronic enhancer (306); Recognition sequence for a site-specific recombinase (307; the sequence of this site is in opposite orientation to the site labeled 308); Recognition sequence for a site-specific recombinase (308; the sequence of this site is in opposite orientation to the site labeled 307); Recognition sequence for a different site-specific recombinase (314; the sequence of this site is in same orientation as the site labeled 315); Constant domain exons (309; in native arrangement, but in antisense orientation relative to V gene segments in the same allele); IgM constant domain exons (310; in native arrangement and in sense orientation relative to V gene segments in the same allele); a selectable element (311) that only expresses a protein product if it is downstream of a productively rearranged V(D)J exon; IgG constant domain exons (312) downstream of a native class switch recombinase sequence (not separately labeled) and upstream of a component (313) of a lineage marking system that can be used to identify cells that have gained antigen experience through use of allele 302; Heterodimerizer elements (316, 317).

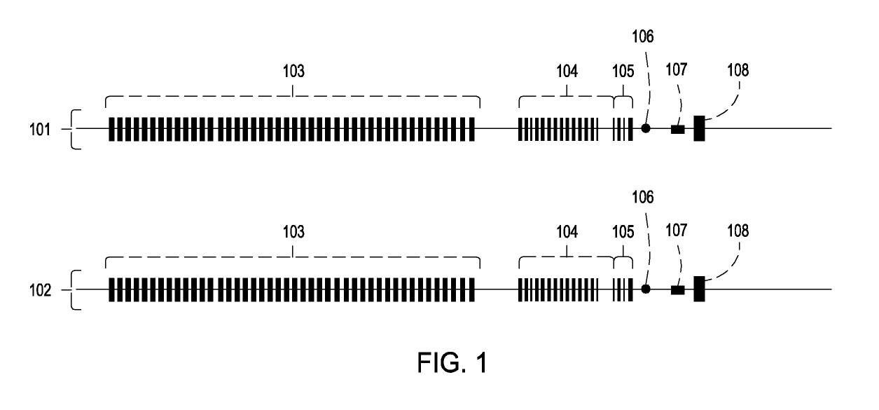

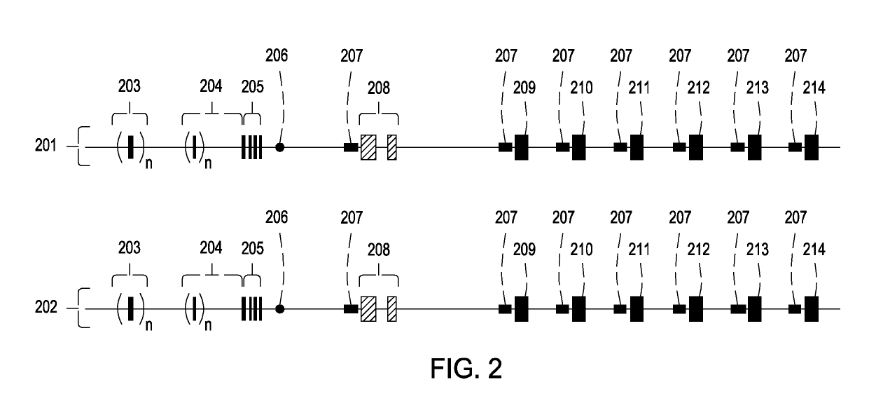

Heavy Chain Allele 301, e.g., comprises the following elements: 5′-VH (303)-DH (304)-JH (305)-Heavy chain intronic enhancer (306)-Site specific recombination site-1 (314)-Site specific recombination site-2 (307)-CD79A (311)-Heavy chain constant region exons inverted (309)-Site specific recombination site-2 (308)-Site specific recombination site-1 (315)-Heterdimerizer-1 (316)-3′; where VH, DH, JH are chimeric genes comprising human coding regions and mouse introns and mouse regulatory sequences as described in US Pub. No. 2013/0219535 to Wabl and Killeen and where the heavy chain enhancer and heavy chain constant region genes comprise a sequence, such as LOCUS: NG_005838 (1..180,971); Site specific recombination site-1 is a site for any site-specific recombinase other than Cre or Flp, e.g., a ROX site for Dre recombinase or an attB/attP site for phiC31 integrase (see, e.g., Anastassiadis K, et al., Disease Models and Mechanisms, 2:508-515 (2009), and Groth A C, et al., Proc. Natl. Acad. Sci. USA., 97:5995-6000 (2000)); Site specific recombination site-2 is, e.g., a loxP site (see, e.g., Oberdoerffer P, et al., Nucleic Acids Res 31:e140 (2003)); CD79A hybrid exon that comprises a splice acceptor, a linker, PTV1-2A peptide, the mouse CD79A open reading frame of the mature protein and the 3′ untranslated region [SEQ ID No. 1]; Heterodimerizer-1, comprising, a sequence such as, e.g., Mus musculus jun proto-oncogene LOCUS: NM_010591 or the mutant C-gamma1 constant region described herein at ¶ [00099].

Heavy Chain Allele 302 comprises, e.g., the following elements: 5′-VH (303)-DH (304)-JH (305)-Heavy Chain Intronic Enhancer (306)-Site specific recombination site-1 (314)-Site specific recombination site-2 (307)-Heavy chain constant region mu (310)-Heavy chain constant region gamma1 (312) and 3′UTR with IRES-FLP element (313)-CD79A (inverted 311)-Site specific recombination site-2 (308)-Site specific recombination site 1 (315)-heterdimerizer-2 (317)-3′; where VH, DH, JH are chimeric genes regulatory sequences as described in US Pub. No. 2013/0219535 to Wabl and Killeen and where the heavy chain enhancer and heavy chain constant region exons and 3′UTR comprise a sequence such as LOCUS: NG_005838 (1..180,971); CD79A hybrid exon that comprises a splice acceptor, a linker, PTV1-2A peptide, the mouse CD79A open reading frame of the mature protein and the 3′ untranslated region such as [SEQ ID No. 1]; Site specific recombination site-1 is a site for any site specific recombinase other than Cre or Flp, e.g., a ROX site for Dre recombinase or an attB/attP site for phiC31 integrase (see, e.g., Anastassiadis K, et al. Disease Models and Mechanisms 2:508-515 (2009), and Groth A C, et al. Proc. Natl. Acad. Sci. USA. 97:5995-6000 (2000)); Site specific recombination site-2 is, e.g., a loxP site (see, e.g., Oberdoerffer P, et al. Nucleic Acids Res 31:e140 (2003)); Heterodimerizer-1, comprising a sequence such as, e.g., Mus musculus FBJ osteosarcoma oncogene (Fos) LOCUS: NM_010591 or the mutant C-gamma1 constant region described herein [00099].

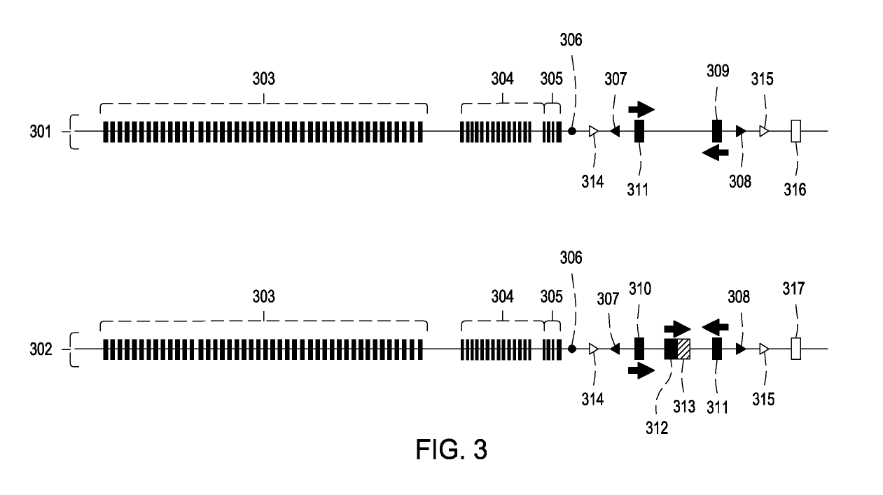

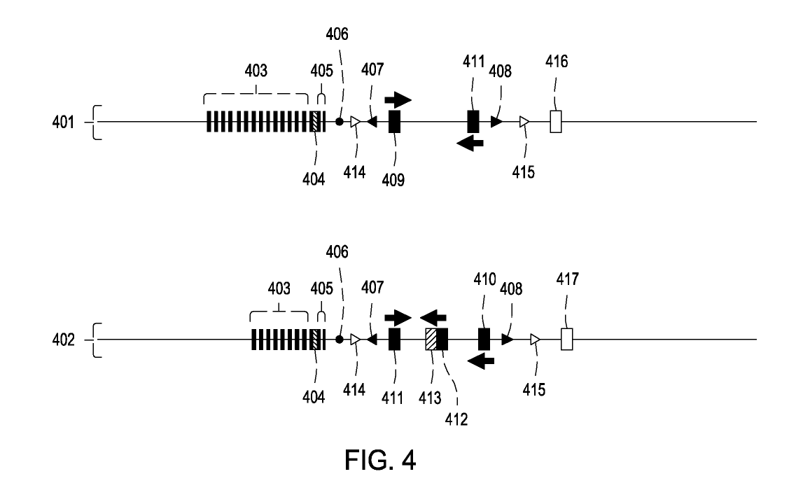

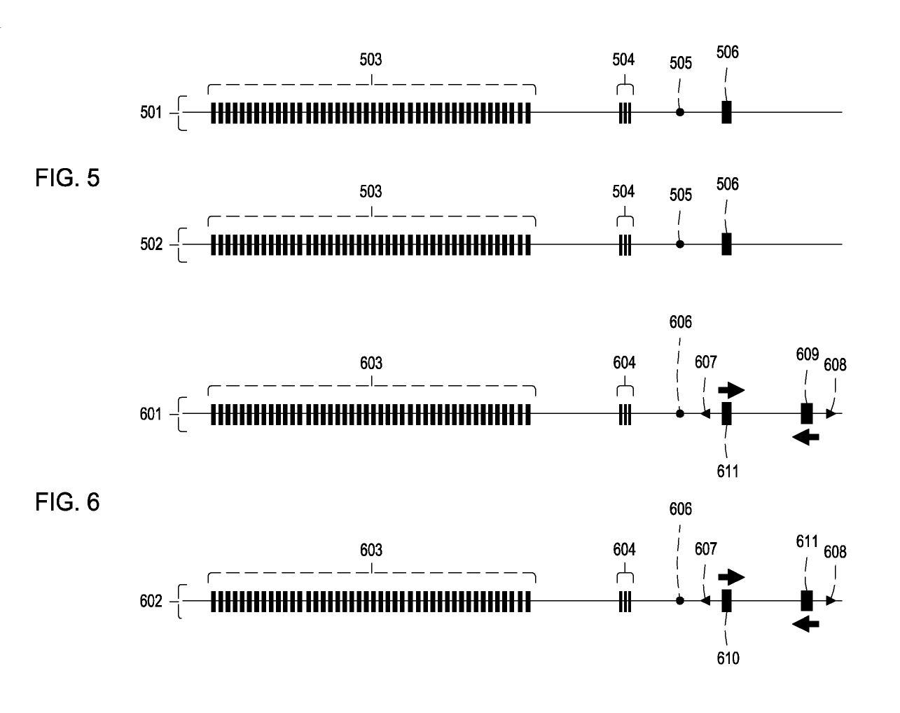

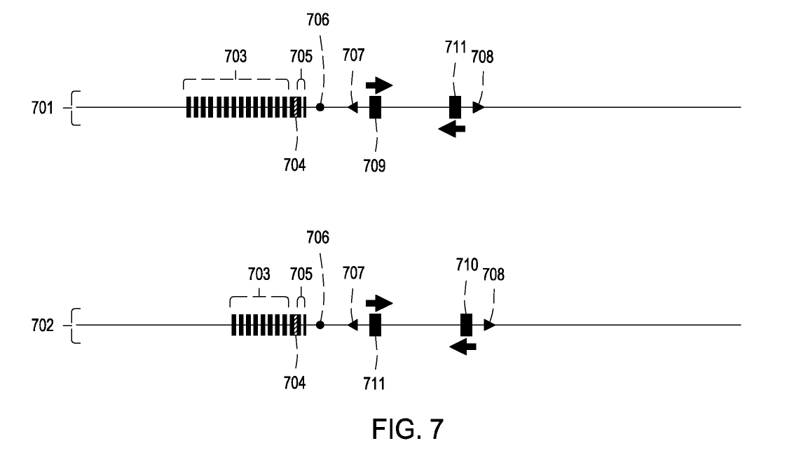

FIG. 4. A combination of heavy chain alleles permissive for isolation of bispecific antibodies featuring a genetic modification to select for allelic inclusion (after VDJ rearrangement and site-specific recombination on both alleles). FIG. 4 depicts the heavy chain alleles found on two homologous chromosomes as they would be found in antigen-experienced B cells in a mouse (i.e., after V(D)J rearrangement and after antigen-driven clonal expansion). These are the same heavy chain alleles as depicted in FIG. 3, except they are shown as they would be configured after VDJ rearrangement and site-specific recombination on both alleles (involving the sites labeled 407 and 408 in this figure, which correspond to the sites labeled 307 and 308 in FIG. 3). The heavy chain alleles (401 and 402) are comprised of chimeric gene segments containing human coding regions and mouse non-coding regions, although other kinds of gene segments are compatible with the invention and can be employed. Again, in this context, as with other alleles described herein, an allele such as 401 and 402 refers to a chromosome segment that may include V, D, and J genes, an intronic enhancer, constant regions genes, and other sequences. After V(D)J rearrangement and appropriate site-specific recombination, allele 401 is now capable of expressing a full-length heavy chain protein because the open reading frame in its constant domain exons (409) is in sense orientation relative to the open reading frame of the VDJ exon (404) in the same allele. By contrast, allele 402 is no longer capable of expressing a full-length heavy chain protein because the open reading frame in its constant domain exons (410 and 412) is now in antisense orientation relative to the open reading frames of the VDJ exon (404) in the same allele. The element (411) in allele 401 that previously allowed for in vivo selection for productive V(D)J rearrangements on this allele is no longer expressed because it is now in antisense orientation relative to the open reading frames of the V gene segments (403) in the same allele. However, the same sort of element (411) on allele 402 is now expressed, permitting ongoing B cell development. Labeled components in the figure are as follows: Variable region gene segments (403); Productively rearranged VDJ exons (404); J region gene segments (405); Heavy chain intronic enhancer (406); Recognition sequence for a site-specific recombinase (407; the sequence of this site is in opposite orientation to the site labeled 408); Recognition sequence for a site-specific recombinase (408; the sequence of this site is in opposite orientation to the site labeled 407); Recognition sequence for a different site-specific recombinase (414; the sequence of this site is in the same orientation as the site labeled 415); Ig constant domain exons (409; in sense orientation relative to the VDJ exon in the same allele); IgM constant domain exons (410; in native arrangement but in antisense orientation relative to the VDJ exon in the same allele); a selectable element (411) that only expresses a protein product if it is downstream of a productively rearranged VDJ exon (and can be the same as element 311 of FIG. 3 such as the sequence in [SEQ ID No. 1]); IgG constant domain exons (412) now upstream of a native class switch recombinase sequence (not separately labeled) and downstream of the component (413) of a lineage marking system that was used to identify cells that previously gained antigen experience through use of allele 302. This component is no longer functional in the depicted configuration after site-specific recombination. Labeled components in the figure are as follows: Heavy Chain Alleles (401, 402); Variable region gene segments (403); Productively rearranged VDJ exons (404); J region gene segments (405); Heavy chain intronic enhancer (406); Recognition sequence for a site-specific recombinase (407; the sequence of this site is in opposite orientation to the site labeled 408); Recognition sequence for a site-specific recombinase (408; the sequence of this site is in opposite orientation to the site labeled 407); Recognition sequence for a different site-specific recombinase (414; the sequence of this site is in the same orientation as the site labeled 415); Recognition sequence for this site-specific recombinase (415; the sequence of this site is in the same orientation as the site labeled 414); Constant domain exons (409; in native arrangement, but in sense orientation relative to the VDJ exon in the same allele); IgM and IgG constant domain exons (410, 412, now in antisense orientation relative to the VDJ exon in the same allele); a selectable element (411); Component (313) of a lineage marking system that was used to identify cells but is now non-functional; Heterodimerizer elements (416 and 417).

FIG. 5. Kappa light chain alleles on two homologous chromosomes in committed lymphoid progenitors in a mouse prior to VJ rearrangement. The light chain alleles (501 and 502) in this embodiment are comprised of chimeric V and J gene segments containing human coding regions and mouse non-coding regions, although other kinds of gene segments are compatible with the invention and can be employed. In this context, as with other alleles described herein, an allele such as 501 and 502 refers to a chromosome segment that may include V and J genes, an intronic enhancer, constant regions genes, and other sequences. Depicted are the V (503) and J (504) gene segments, which undergo recombination during B cell development, the light chain intronic enhancer (505) involved in transcription of the L chain locus, and the constant domain exon (Cκ, 506). Labeled components in the figure are as follows: Chromosomal segments at the light chain locus (501, 502); Variable region gene segments (503); J region gene segments (504); Light chain intronic enhancer (505); Constant domain exon (506).

FIG. 6. A combination of light chain alleles permissive for isolation of bispecific antibodies featuring a genetic modification to select for allelic inclusion. FIG. 6 depicts the light chain alleles found on two homologous chromosomes in committed lymphoid progenitors in a mouse (i.e., prior to light chain VJ gene rearrangement). The light chain alleles (601 and 602) comprise chimeric gene segments containing human coding regions and mouse non-coding regions, although other kinds of gene segments are compatible with the invention and can be employed. In this context, as with other alleles described herein, an allele such as 601 and 602 refers to a chromosome segment that may include V, D, and J genes, an intronic enhancer, constant regions genes, and other sequences, including elements 603 to 611, described below. After VJ rearrangement, but prior to appropriate site-specific recombination, allele 601 is not capable of expressing a full-length light chain protein because the open reading frame of its constant domain exon (609) is in an antisense orientation relative to the open reading frames of the V gene segments (603) in the same allele. By contrast, allele 602 is capable of expressing a full-length light chain protein because the open reading frame of its constant domain exon (610) is in a sense orientation relative to the open reading frames of the V gene segments (603) in the same allele. Allele 601 includes an element (611) that allows for in vivo selection for productive VJ rearrangements on this allele; translation of this element only occurs if an upstream rearranged VJ exon contains a translated unbroken open reading frame, and the protein product of the 611 element is required for survival of B cells once they leave the bone marrow where they are generated. Allele 602 also includes the same sort of element (611), but in inverted translational orientation relative to the open reading frames in the V gene segments in the same allele. Because of this inverted orientation, the element is not translated until after site-specific recombination (involving sites 607 and 608). Labeled components in the figure are as follows: Chromosomal segments at light chain loci (601, 602); Variable region gene segments (603); J region gene segments (604); Light chain intronic enhancer (606); Site-specific Recombination Sites (607, 608); Constant Domain Exon in antisense orientation (609); Constant Domain Exon in sense orientation (610); Element that allows for in vivo Selection for Productive VJ Rearrangements (611).

Light Chain Allele 601 comprises, e.g., the following elements: 5′-VK-JK-K intron enhancer-loxP site-BAFF-R-C-Kappa inverted-loxP site-3′; where VK and JK are chimeric genes comprising human coding regions and mouse introns and mouse regulatory sequences as described in US Pub. No. 2013/0219535 to Wabl and Killeen and comprising sequences for a light chain enhancer and light chain constant region gene, such as e.g., sequence NC_000072.6 (67555636..70726754); and a BAFF-R hybrid exon comprising a splice acceptor, a linker, a PTV1-2A peptide, the mouse Tnfrsf13c open reading frame, and a 3′ untranslated region such as [SEQ ID No. 2].

Light Chain Allele 602 comprises, e.g., the following elements: 5′-VK-JK-K intron enhancer-loxP site-C-Kappa inverted-BAFF-R-loxP site-3′; where VK and JK are chimeric genes consisting of human coding regions and mouse introns and regulatory sequences as described in US Pub. No. 2013/0219535 to Wabl and Killeen and comprising sequences for a light chain enhancer and light chain constant region gene, e.g., NC_000072.6 (67555636..70726754); and a BAFF-R hybrid exon comprising a splice acceptor, a linker, a PTV1-2A peptide, the mouse Tnfrsf13c open reading frame, and a 3′ untranslated region such as [SEQ ID No. 2].

FIG. 7. A combination of light chain alleles permissive for isolation of bispecific antibodies featuring a genetic modification to select for allelic inclusion (after VJ rearrangement and site-specific recombination on both alleles). FIG. 7 depicts the light chain alleles found on two homologous chromosomes as they would be found in antigen-experienced B cells in a mouse (i.e., after VJ rearrangement and after antigen-driven clonal expansion). These are the same light chain alleles as depicted in FIG. 6, except they are shown as they would be configured after VJ rearrangement and site-specific recombination on both alleles (involving the sites labeled 707 and 708 in this figure, which correspond to the sites labeled 607 and 608 in FIG. 6). The light chain alleles (701 and 702) are comprised of chimeric gene segments containing human coding regions and mouse non-coding regions, although other kinds of gene segments would be compatible with the invention and thus could also be employed. In this context, as with other alleles described herein, an allele such as 701 and 702 refers to a chromosome segment that may include V, D, and J genes, an intronic enhancer, constant regions genes, and other sequences. After VJ rearrangement and appropriate site-specific recombination, allele 701 is now capable of expressing a full-length light chain protein because the open reading frame in its constant domain exon (709) is in sense orientation relative to the open reading frame of the VJ exon (704) in the same allele. By contrast, allele 702 is no longer capable of expressing a full-length heavy chain protein because the open reading frame in its constant domain exon (710) is now in antisense orientation relative to the open reading frame of the VJ exon (704) in the same allele. The element (711) in allele 701 that previously allowed for in vivo selection for productive VJ rearrangements on this allele is no longer expressed because it is now in antisense orientation relative to the open reading frames of the VJ exon (703) in the same allele. However, the same sort of element (711) on allele 702 is now expressed, permitting ongoing B cell survival (element 711 can be the same as element 611 of FIG. 6 [SEQ ID No. 2]). Labeled components in the figure are as follows: Variable region gene segments (703); productively rearranged VJ exons (704); J gene segments (705); Light chain intronic enhancer (706); Recognition sequence for a site-specific recombinase (707; the sequence of this site is in opposite orientation to the site labeled 708); Recognition sequence for a site-specific recombinase (708; the sequence of this site is in opposite orientation to the site labeled 707); Ig L chain constant domain exon (709; in sense orientation relative to the VJ exon in the same allele); L chain constant domain exon (710; in antisense orientation relative to the VJ exon in the same allele); a selectable element (711) that only expresses a protein product if it is downstream of a productively rearranged VJ exon.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “transgene” is used herein to describe genetic material which has been or is about to be artificially inserted into the genome of a cell, and particularly a cell of a vertebrate host animal.

By “transgenic animal” it is meant a non-human animal, usually a mammal, such as a rodent, particularly a mouse or a rat though other mammals are envisioned as well, having an exogenous nucleic acid sequence present as a chromosomal or extrachromosomal element in a portion of its cells or stably integrated into its germ line DNA (i.e., in the genomic sequence of most or all of its cells).

A “vector” includes plasmids and viruses and any DNA or RNA molecule, whether self-replicating or not, which can be used to transform or transfect a cell.

“Cell surface” refers to the plasma membrane of the cell, i.e., that part of the cell most directly exposed to extracellular spaces and available for contact both with cells and proteins in the extracellular (including intercellular) space.

A “bispecific antibody” is one that comprises two physically separable antigen-binding surfaces which differ from one another in their antigen specificity. Normal IgG antibodies have two physically separable antigen-binding surfaces that are structurally identical and thus have the same antigen specificity. A preferred version of a bispecific antibody is one that resembles a normal IgG antibody molecule with two physically separable antigen-binding surfaces, but instead of these surfaces being structurally identical they differ from each other. Both of these surfaces may be comprised of the same heavy chain protein but differ from each other in the light chain proteins they comprise. Alternatively, the two surfaces may be comprised of the same light chain protein but differ from each other in the heavy chain proteins they comprise.

“Allelic exclusion” refers to the fact that the vast majority of B cells in vertebrate species such as rodents or humans carry a productively rearranged heavy chain gene on only one of two homologous autosomes. Allelic exclusion at light chain loci would refer to an analogous scenario. In a more general sense, allelic exclusion applies whenever productive V(D)J rearrangement at any heavy or light chain locus inhibits further rearrangement of other heavy or light chain V(D)J gene segments, respectively, no matter where their chromosomal location. For example, if two or more sets of heavy chain VDJ linkage groups are inserted in the same chromosome, productive rearrangement at one of the heavy chain linkage groups prevents further V(D)J rearrangement at any of the other heavy chain linkage groups. The same would apply to light chain linkage groups. In principle this type of “allelic” exclusion would occur by the same mechanism as conventional allelic exclusion

“Allelic inclusion” refers to a loss of allelic exclusion, and thus to an increased representation of B cells with productive V(D)J rearrangements on both alleles of the heavy and/or light chain loci.

As used herein, “productive rearrangement” is a VDJ or VJ rearrangement that is in frame with the constant domain exon(s) and enables translation of a full length heavy or light chain protein containing variable and constant region domains. The productive VDJ or VJ rearrangement can also be in frame with a heterologous exon that replaces the constant domain exon(s).

A genomic “locale” is any region of the genome, preferably a gene, which is associated with one particular functional aspect. The term locale is used here to refer to parts of immunoglobulin loci. For example, locale can refer to that part of an immunoglobulin locus that primarily contains one kind of gene segment, such as a V gene segment locale, or a D gene segment locale, or a J gene segment locale, or more broadly, the variable locale, which includes all of the V, D and J gene segments. The constant region locale is that part of an immunoglobulin locus that contains constant region exons.

As used herein, “homodimer” refers to an IgG or other isotype antibody comprising two identical heavy (H) chains, where each of the H chains is paired with an identical light (L) chain. As used herein, “heterodimer” refers to an IgG or the isotype antibody comprising two heavy (H) chains, where the H chains may or may not be identical, and where each of the H chains is paired with a light (L) chain, where each of the L chains may or may not be identical; however in a heterodimer, if the H chains are identical, the L chains will be different from one another, and if the L chains are identical, the H chains will be different from one another.

### DETAILED DESCRIPTION OF THE INVENTION

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999) Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Lehninger, Principles of Biochemistry 3^{rd} Ed., W. H. Freeman Pub., New York, N.Y.; and Berg et al. (2002) Biochemistry, 5^{th} Ed., W.H. Freeman Pub., New York, N.Y.; Nagy, et al., Eds. (2003) Manipulating the Mouse Embryo: A Laboratory Manual (3^{rd} Ed.) Cold Spring Harbor Laboratory Press, Cold Spring Harbor, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “an immunoglobulin” refers to one or more such immunoglobulins, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention.

### The Invention in General

One embodiment of the invention, a transgenic animal (typically a mammal, and more typically a rodent) carries engineered versions of its heavy chain locus on both chromosomes. Both engineered heavy chain alleles are capable of undergoing V(D)J rearrangement to create heavy chain diversity during B cell development. One of the engineered heavy chain alleles is also capable of expressing a full-length transmembrane heavy chain protein that can generate a pre-BCR signal. The other engineered heavy chain allele is disabled in this regard.

The engineered heavy chain alleles in the embodiment of the invention just summarized are comprised of mutations in the part of the gene that encodes the constant domain of the heavy chain molecules.

In a further embodiment of the invention, the two engineered heavy chain alleles carry recognition sequences (wild-type or mutated) for one or more site-specific recombinases such as Cre or Flp. The sites are placed in such a way that site-specific recombination would change the functionality of the constant domain-encoding part of the gene. That is, if the allele is capable of expressing a fully functional heavy chain protein, then site-specific recombination would deprive the allele of this property. Similarly, if the allele is incapable of expressing a fully functional heavy chain protein, then site-specific recombination would confer this property on the allele.

The site-specific recombinase-mediated changes just summarized may be accomplished either by deleting or inverting pieces of DNA in the constant domain-encoding part of the two homologous heavy chain alleles.

In a preferred embodiment of the invention, site-specific recombinase-dependent loss of constant domain full functionality on one chromosome would be accompanied by synchronous, or near synchronous, gain of full functionality on the other chromosome.

In a further preferred embodiment of the invention, the transgenic animals just described are immunized with an antigen allowing for clonal expansion of B cells expressing antibody molecules specific for the antigen. Because of the heavy chain gene mutations the B cells carry, the antibodies specific for this antigen are comprised of heavy chains encoded by rearranged versions of only one of the two heavy chain alleles; namely, the allele defined by full functionality in its constant domain-encoding part. The other allele lacks such functionality and because of this does not encode heavy chains capable of participating in the signaling process necessary for antigen-specific clonal expansion.

In a variation of the embodiment just mentioned, repeated immunizations are employed to maximize clonal expansion and antigen-specific antibody diversity.

After the immunization regimen has been completed, site-specific recombination is induced resulting in a switch of heavy chain constant domain functionality from one allele to the other. As a result of this switch, the allele encoding the heavy chains in antibodies specific for the antigen used in the immunization is deprived of constant domain full functionality. At the same time, or near to it, the allele that was previously deprived of constant domain full functionality now gains this functionality. Through this switch in functionality, cells that participated in clonal expansion in response to the antigen used in the immunization regimen gain expression of new heavy chain proteins.

Subsequent to the induced site-specific recombinase-dependent switch just described—and in a further embodiment of the invention—the animals are immunized with a second antigen.

Clonal expansion in response to the second antigen depends on antibodies comprised of heavy chains encoded by the second allele, i.e., the one that gained constant domain functionality due to the induced site-specific recombinase event.

A second immunization may be repeated to maximize clonal expansion and antigen-specific antibody diversity.

In another embodiment of the invention, clonally expanded cells expressing antibodies specific for the second antigen are those that had previously been involved in clonal expansion in response to the first antigen. During the second clonal expansion, these cells do not express the heavy chain protein that conferred specificity for the first antigen but instead express a different heavy chain protein. The heavy chain protein they express that confers specificity for the second antigen is encoded by the heavy chain allele that gained constant domain full functionality as a consequence of the induced site-specific recombination event.

After the second immunization regimen has been completed, hybridoma or other techniques are employed to isolate antigen-specific B cells.

In one embodiment of the invention, B cells specific for the second antigen are isolated using hybridoma or other technology. In an alternative embodiment of the invention, a second site-specific recombinase event is induced before isolation of B cells specific for the first antigen using hybridoma or other technology.

In a further embodiment of the invention, B cells, hybridomas or other derivatives specific for one of the two antigens are modified by site-specific recombination, or another means. This modification confers constant domain full functionality on the allele they carry that previously lacked full functionality. Through this modification it is possible to assess the capacity of the cells to express two types of antibody molecules: one specific for the antigen used in the first immunization regimen, and the other specific for the antigen used in the second immunization regimen.

In another embodiment of the invention, pairs of proteins or protein domains, termed heterodimerizers, are used to induce heterodimerization and suppress homodimerization of the antigen binding domains encoded by the two heavy chain alleles of the bispecific B cells, hybridomas or other derivatives. One member of each heterodimerizer pair is encoded by each heavy chain allele and replaces the normal constant domains of the antibody heavy chain. The heterodimerizers in one embodiment are pairs of IgG dimerization domains that are each mutated so that they preferentially form heterodimers when present in a population of two different IgG heavy chains, and in another embodiment are non-immunoglobulin proteins such as c-Fos and c-Jun, which physiologically heterodimerize to form the AP-1 transcription factor, or other leucine zipper-type proteins or protein domains.

In yet a further embodiment of the invention, rather than allelic exclusion being compromised at the heavy chain locus, allelic exclusion is instead compromised at a light chain locus. In this version of the invention, heavy chain allelic exclusion would be normal. The light chain allelic inclusion version of the invention is conceptually similar to that of the heavy chain allelic inclusion version, featuring analogous mutations in the constant domain-encoding part of the relevant homologous light chain genes. Light chain allelic inclusion is exploited using a similar double immunization scheme combined with an appropriately staged inducible site-specific recombination step. Bispecific B cells are identified and/or isolated in a similar fashion to what has been described for the heavy chain allelic inclusion version of the invention. This light chain allelic inclusion version of the invention is expected to yield bispecific antibodies each comprised of two light chain proteins and one heavy chain protein, whereas the heavy chain allelic inclusion version would yield bispecific antibodies each comprised of one light chain protein and two heavy chains.

### Transgenic Cell Libraries

The transgenic cells of the invention may also be used to produce expression libraries, preferably low complexity libraries, for identification of antibodies of interest on the surface of plasma cells. The present invention thus also includes antibody libraries produced using the cell technologies of the invention for identification of antigen-specific antibodies expressed on plasma cells.

### Transgenic Animals

In specific aspects of the invention, the invention provides transgenic animals carrying engineered immunoglobulin heavy chain or light genes.

In a preferred aspect, the transgenic animals of the invention further comprise human immunoglobulin regions. For example, numerous methods have been developed for replacing endogenous mouse immunoglobulin regions with human immunoglobulin sequences to create partially- or fully-human antibodies for drug discovery purposes. Examples of such mice include those described in, for example, U.S. Pat. Nos. 7,145,056; 7,064,244; 7,041,871; 6,673,986; 6,596,541; 6,570,061; 6,162,963; 6,130,364; 6,091,001; 6,023,010; 5,593,598; 5,877,397; 5,874,299; 5,814,318; 5,789,650; 5,661,016; 5,612,205; and 5,591,669.

In a particularly preferred aspect, the transgenic animals of the invention comprise chimeric immunoglobulin segments as described in co-pending application US Pub. No. 2013/0219535 by Wabl and Killeen. Such transgenic animals have a genome comprising an introduced partially human immunoglobulin region, where the introduced region comprising human variable region coding sequences and non-coding variable sequences based on the endogenous genome of the non-human vertebrate. Preferably, the transgenic cells and animals of the invention have genomes in which part or all of the endogenous immunoglobulin region is removed.

### Use in Antibody Production

Culturing cells in vitro has been the basis of the production of numerous therapeutic biotechnology products, and involves the production of protein products in cells and release into the support medium. The quantity and quality of protein production over time from the cells growing in culture depends on a number of factors, such as, for example, cell density, cell cycle phase, cellular biosynthesis rates of the proteins, condition of the medium used to support cell viability and growth, and the longevity of the cells in culture. (See, for example, Fresney, Culture of Animal Cells, Wiley, Blackwell (2010); and Cell Culture Technology for Pharmaceutical and Cell-Based Therapies, Ozturk and Ha, Eds., CRC Press, (2006).)

The invention provides a source of B cells derived from immunization schemes in which animals are first provided with one antigen and then later with another antigen. In both cases, multiple immunizations may be employed to increase antigen-specific antibody titers in individual animals. An inducible site-specific recombination step would be included between the two immunization series. Subsequent to the second immunization scheme, B cells are isolated and cultured or used to create hybridomas, or used as a source of RNA for cloning immunoglobulin chain genes. The B cells or the antibody molecules they contain are tested for bispecific antigen-binding properties. In the case of hybridomas, this is accomplished by screening hybridomas for specificity for one kind of antigen, and then analyzing them for whether they carry additional rearranged immunoglobulin chain genes that confer specificity for a second kind of antigen, i.e., they have latent or expressed bi-specificity.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to terms and numbers used (e.g., vectors, amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1: Engineered Heavy Chain Alleles Permissive for the Isolation of Bispecific Antibodies Featuring a Genetic Modification to Select for Allelic Inclusion

Mice are generated carrying heavy chain alleles that differ from one another in their constant domain-encoding locales. One of the alleles is capable of expressing a full length heavy chain protein after a productive V(D)J rearrangement. The other allele lacks this functionality.

Both alleles contain recognition sequences for two different site-specific recombinases, positioned within the constant domain-encoding locale. Site-specific recombination at one of these sites causes an inversion of a piece of DNA in both alleles, and as a consequence of this inversion, the constant domain functionality in the alleles is changed. A subsequent site-specific recombination in bispecific B cells by a different recombinase causes replacement of the normal constant domains of the immunoglobulin heavy chains with heterodimerizers, pairs of proteins or protein domains that favor heterodimerization over homodimerization of the two different heavy chains produced by these B cells. Also included are elements designed to improve the efficiency with which the desired kind of bispecific B cells are isolated.

Site-specific recombination confers the capacity to express a full-length heavy chain protein on the allele that initially lacked this capacity. By contrast, site-specific recombination removes this capacity from the allele that initially had it.

One version of this kind of paired alleles is depicted in FIG. 3, with the site-specific recombination sequences relevant for conferring allele functionality marked as 307 and 308, and with the constant domain-encoding exons labeled as 309 and 310. The transcriptional orientation of the constant domain-encoding exons is signified by the arrow immediately below the labeled components. FIG. 3 shows the two alleles in their germline configuration before V(D)J rearrangement has occurred.

V(D)J rearrangement of the alleles depicted in FIG. 3 may result in nonproductive (i.e., out-of-frame) gene segment joins on either allele. Similarly, the process may also result in productive (i.e., in-frame) rearrangements on either allele. B cells only complete their development in the bone marrow if they express a full-length heavy chain molecule that is capable of generating signals at the plasma membrane as part of a pre-BCR or an immunoglobulin light chain-containing BCR. Using the combination of alleles depicted in FIG. 3, such a full-length heavy chain protein could only derive from allele 302. This is because even though allele 301 can undergo a productive V(D)J rearrangement it cannot express a full-length heavy chain protein.

Element 311 is designed to provide selection for in-frame rearrangements on the allele (301) that is not capable of expressing a full-length heavy chain protein. For example, one embodiment of this element is one that begins with a sequence encoding a self-cleaving (2A) peptide fused in-frame with an open reading frame for a protein that is crucial for pre-BCR and BCR signaling, such as the CD79a protein (R. Pelanda, et al. [2002] Journal of Immunology 169:865-872). If a mouse is employed that is homozygous for a loss-of-function mutation in the endogenous gene encoding its CD79a protein, then the only source of this protein in B cells is the 311 element. The 2A self-cleaving peptide sequence is placed such that it would be in-frame with a productively rearranged VDJ exon. If the 301 allele fails to rearrange productively, no CD79a protein is expressed and B cell development is blocked. B cells are only capable of completing development if they undergo an in-frame rearrangement and thereby gain expression of CD79a.

Crucially, the selection scheme just outlined rescues CD79a protein expression, but B cells only develop if they also undergo a productive rearrangement on their 302 allele and thereby gain expression of a full-length heavy chain protein.

B cell receptor signaling during a response to an antigen also requires CD79a. A deficiency of CD79a therefore blocks signaling not only in the bone marrow but also in mature B cells. Thus, the 311 element also provides selection for B cells to continue to retain productive rearrangements on both of their alleles so that normal homeostasis is preserved and so that B cells are capable of participating in immune responses.

Peripheral B cells that develop in the mice carrying the alleles shown in FIG. 3 express B cell antigen receptors (transmembrane or secreted) comprised of heavy chains derived from allele 302. The light chains in these B cell antigen receptors derive from normal independent VJ rearrangements at their light chain loci, and in one embodiment of this invention the light chain loci are engineered such that they carry chimeric gene segments comprised of human coding regions and mouse noncoding regions. Other kinds of light chain loci are also compatible with the invention.

Immunization of mice carrying the alleles shown in FIG. 3 results in clonal expansion of B cells expressing antibodies specific for the antigen used in the immunization. Crucially, once again, these antibodies are solely comprised of heavy chains derived from allele 302. Repeated immunizations are expected to result in enhanced clonal expansion, somatic hypermutation, and enhanced antigen-specific antibody diversity, similar to what occurs in normal mice.

Transgenic mouse systems exist, or are readily engineered, to permit inducible expression of particular site-specific recombinases in multiple cell types including B lymphocytes. Immunized mice that have made demonstrable antibody responses to the antigen used in the immunization are caused to express the relevant site-specific recombinase, obviously requiring that the mice carry in them the necessary inducible site-specific recombinase system.

Induced site-specific recombination at the rearranged versions of the germline alleles depicted in FIG. 3 give rise to the sort of outcome depicted in FIG. 4. One key feature evident in FIG. 4 is a loss on allele 402 (i.e., the rearranged version of allele 302 after site-specific recombination) of the capacity to express a full-length heavy chain protein. Similarly, there is a gain of this capacity on allele 401, which is the rearranged version of allele 301 after site specific recombination. The 411 element is no longer expressed from allele 401 following site-specific recombination because of its inverted orientation, but this element is also present in an analogous location on allele 402. Thus, e.g., as B cells lose expression of CD79a from allele 401 subsequent to site-specific recombination, they gain it from allele 402 if this allele also undergoes recombination. The mirrored/reciprocal placement of the element on both alleles creates a selection for both alleles undergoing near-synchronous site-specific recombination, as desired for the isolation of bispecific antibodies.

Immunization of mice carrying the arrangement of alleles depicted in FIG. 4 result in clonal expansion of B cells expressing B cell antigen receptors specific for the antigen used in the immunization. These B cell antigen receptors (transmembrane or secreted) are comprised solely of heavy chains derived from allele 401. Repeated immunizations are expected to result in enhanced clonal expansion, somatic hypermutation, and enhanced antigen-specific antibody diversity, similar to what occurs in normal mice.

B cells specific for the antigen used in the second immunization include some B cells that have not undergone clonal expansion in response to the first antigen. Such B cells are not an obvious source for bispecific antibodies capable of recognizing the antigens used in the two immunizations. However, some fraction of the B cells specific for the second antigen have been involved in clonal expansion in response to the first antigen. These B cells are an obvious source for bispecific antibodies: with one of their rearranged heavy chain genes they produce antibodies that have specificity for one of the two antigens, while with their other rearranged heavy chain gene they produce antibodies specific for the other antigen. In both cases, individual B cells would use one light chain protein to pair with both heavy chain proteins.

The 312 element is a set of IgG constant domain-encoding exons with an associated upstream switch recombination sequence. The presence of this element in the 302 allele confers on the allele the capacity to undergo class switch recombination from IgM to IgG. A switch to IgG isotype usage facilitates the isolation of B cells expressing antibodies with high affinity for an antigen. It also permits a marking system to be included in the allele such as is represented by the element labeled as 313.

A possible embodiment of element 313 is for this to be comprised of an IRES-Flp element. Cells that undergo an IgM to IgG class switch recombination gain expression of the Flp recombinase through the IRES-Flp 313 element placed within the 3′ untranslated region of the last IgG exon.

Systems exist, or can be readily engineered, that allow for cells to gain essentially irreversible expression of a reporter due to Flp recombination. For example, if the mouse carrying the 301 and 302 alleles also carries such a system (e.g., a Rosa26-FRT-Stop-FRT-YFP reporter transgene), then B cells that have undergone class switch recombination to IgG on the 302 allele gain Flp expression and thereby irreversibly gain reporter (e.g., YFP) expression.

Marking cells that have undergone class switch recombination during an immunization permits the isolation of these cells prior to, or after, the induced site-specific recombination step and/or a second immunization step. If the cells are isolated prior to the second immunization step, they may be adoptively transferred into other mice, which could then subsequently be immunized. If the cells are isolated after the second immunization step, their reporter phenotype may be exploited to facilitate the isolation of bispecific cells.

FIG. 4 depicts the alleles of FIG. 3 after V(D)J rearrangement and after induced site-specific recombination. Allele 401 is capable of expressing full-length heavy chain proteins, whereas allele 402 has lost this capability. Allele 401 has lost the ability to express the CD79a protein (411), but allele 402 has gained it. If class switch recombination has occurred (not depicted) then element 413 would have been expressed prior to site-specific recombination and consequently such class-switched cells gain expression of a reporter facilitating their identification and/or isolation.

Hybridoma or other cloning technology may be exploited to recover B cells with specificity for the second immunizing antigen. These B cells are then analyzed to determine whether they also carry a second rearranged heavy chain gene that confers specificity for the second antigen.

Bispecific B cells have different VDJ rearrangements on each heavy chain allele to enable recognition of two antigens. To form a bispecific antibody the two different heavy chains in the cell should form a heterodimer which then binds a common light chain. However, homodimer formation is also possible and this would result in a monospecific antibody. The heavy chain alleles in this example contain exons encoding heterodimerizer elements (416 and 417), which are proteins or protein domains that favor heterodimer over homodimer formation. A possible embodiment of element 416 is c-Jun and of 417 is c-Fos. Site-specific recombination involving the recognition sequences 414 and 415 is used to remove DNA elements upstream of elements 416 and 417. Transcripts now have the structure 5′-VDJ-c-Jun-3′ from allele 401 and 5′-VDJ-c-Fos-3′ from allele 402 and encode fusion proteins that preferentially form heterodimers.

Another possible embodiment of elements 416 and 417 is modification of the IgG1 C_{H}3 dimerization domains on both alleles so that the encoded heavy chains are less compatible for homodimerization but complementary to each other for heterodimerization. The fourth exons of both IgG1 alleles, which encode the C_{H}3 domains, are mutated such that they promote the formation of heavy chain heterodimers and suppress homodimerization. In preferred methods, the mutations are D276K, E233K, and Q234K on one heavy chain allele; and K286D, K269D, and T247D on the other heavy chain allele (amino acid numbering starts at the first codon of C_{H}1). The mutations at similar positions in the human IgG1 heavy chain have been shown to promote heterodimerization and the secretion of bispecific antibodies in cell lines (see, e.g., Gunasekaran, et al., Journal of Biological Chemistry 285:19637-19646 (2010)).

### Example 2: Engineered Light Chain Alleles Permissive for the Isolation of Bispecific Antibodies Featuring a Genetic Modification to Select for Allelic Inclusion

The same general principles used to favor allelic inclusion of the heavy chain locus in Example 1 are utilized here to favor allelic inclusion of the light chain locus.

Mice are generated carrying light chain alleles that differ from one another in their constant domain-encoding locales. One of the alleles is capable of expressing a full length light chain protein after a productive VJ rearrangement. The other allele lacks this functionality.

Both alleles contain recognition sequences for one or more site-specific recombinases positioned within the constant domain-encoding locale. Site-specific recombination at these sites causes an inversion of a piece of DNA in both alleles, and as a consequence of this inversion, the constant domain functionality in the alleles is changed. Also included are elements designed to improve the efficiency with which the desired kind of bispecific B cells is isolated.

Site-specific recombination confers the capacity to express a full-length light chain protein on the allele that initially lacked this capacity. By contrast, site-specific recombination removes this capacity from the allele that initially had it.

One version of this kind of paired alleles is depicted in FIG. 6, with the relevant site-specific recombination sequences marked as 607 and 608, and with the constant domain-encoding exons labeled as 609 and 610. The transcriptional orientation of the constant domain-encoding exon is signified by the arrow immediately above or below the labeled components. FIG. 6 shows the two alleles in their germline configuration before VJ rearrangement has occurred.

VJ rearrangement of the alleles depicted in FIG. 6 results in either nonproductive (i.e., out-of-frame) or productive (i.e., in-frame) rearrangements on either allele. B cells only complete their development in the bone marrow if they express a full-length light chain molecule that is capable of pairing with the immunoglobulin heavy chain and generating signals at the plasma membrane as part of the BCR. Using the combination of alleles depicted in FIG. 6, such a full-length light chain protein only derives from allele 602. This is because even though allele 601 can undergo a productive VJ rearrangement it cannot express a full-length light chain protein.

Element 611 is designed to provide selection for in-frame rearrangements on the allele (601) that is not capable of expressing a full-length light chain protein. One embodiment of this element is one that begins with a sequence encoding a self-cleaving (2A) peptide fused in-frame with an open reading frame for a protein that is crucial for B cell survival in the periphery, such as the receptor for B cell activation factor from the tumor necrosis factor family (BAFF, also known as BLyS, TALL-1, zTNF4 and THANK) (J. S. Thompson et al. [2001] Science, 293:2108-2111; Y. Sasaki et al. [2004] Journal of Immunology 173:2245-2252). The BAFF receptor (BAFF-R) is encoded by the Tumor Necrosis Factor Receptor Superfamily Member 13C gene (Tnfrsf13c). If the mouse used is homozygous for a loss-of-function mutation in the endogenous gene encoding its BAFF-R protein, then the only source of this protein in B cells is the 611 element. The 2A self-cleaving peptide sequence is placed such that it is in-frame with a productively rearranged VJ exon. If the 601 allele fails to rearrange productively, no BAFF-R protein is expressed and the B cell dies shortly after leaving the bone marrow. B cells are only capable of completing their maturation in the periphery if they undergo an in-frame VJ rearrangement and thereby gain expression of BAFF-R.

Crucially, the selection scheme just outlined rescues BAFF-R protein expression, but B cells only mature if they also undergo a productive VJ rearrangement on their 602 alleles and thereby gain expression of a full-length light chain protein.

Survival of mature B cells in the periphery is highly dependent on BAFF-R. Thus, the 611 element provides selection for B cells to continue to retain productive rearrangements on both of their alleles so that normal homeostasis is preserved and so that B cells are capable of participating in immune responses.

Peripheral B cells that develop in the mice carrying the alleles shown in FIG. 6 express B cell antigen receptors (transmembrane or secreted) comprised of light chains derived from allele 602. The heavy chains in these B cell antigen receptors derive from normal independent VDJ rearrangements at their heavy chain locus, and in a preferred embodiment of this invention the heavy chain locus is engineered such that each allele carries chimeric gene segments comprised of human coding regions and mouse noncoding regions. Other kinds of heavy chain loci are also compatible with the invention.

Immunization of mice carrying the alleles shown in FIG. 6 results in clonal expansion of B cells expressing antibodies specific for the antigen used in the immunization. Crucially, once again, the light chains of these antibodies are solely derived from allele 602. Repeated immunizations result in enhanced clonal expansion, somatic hypermutation, and enhanced antigen-specific antibody diversity, similar to what occurs in normal mice.

Transgenic mouse systems exist, or can be readily engineered, to permit inducible expression of particular site-specific recombinases in multiple cell types including B lymphocytes. Immunized mice that have made demonstrable antibody responses to the antigen used in the immunization are caused to express the relevant site-specific recombinase, which obviously requires that the mice carry in them the necessary inducible site-specific recombinase system.

Induced site-specific recombination at the rearranged versions of the germline alleles depicted in FIG. 6 give rise to the sort of outcome depicted in FIG. 7. One key feature evident in FIG. 7 is a loss on allele 702 (i.e., the rearranged version of allele 602 after site-specific recombination) of the capacity to express a full-length light chain protein. Similarly, there is a gain of this capacity on allele 701, which is the rearranged version of allele 601 after site specific recombination. The 711 element is no longer expressed from allele 701 following site-specific recombination because of its inverted orientation, but this element is also present in an analogous location on allele 702. Thus, as B cells lose expression of BAFF-R from allele 701 subsequent to site-specific recombination, they gain it from allele 702 if this allele also undergoes recombination. The mirrored/reciprocal placement of the element on both alleles creates a selection for both alleles undergoing near-synchronous site-specific recombination, as desired for the isolation of bispecific antibodies.

Immunization of mice carrying the arrangement of alleles depicted in FIG. 7 results in clonal expansion of B cells expressing B cell antigen receptors specific for the antigen used in the immunization. The light chains of these B cell antigen receptors (transmembrane or secreted) are solely derived from allele 701. Repeated immunizations are expected to result in enhanced clonal expansion, somatic hypermutation, and enhanced antigen-specific antibody diversity, similar to what occurs in normal mice.

B cells specific for the antigen used in the second immunization would include some that had not undergone clonal expansion in response to the first antigen. Such B cells are not an obvious source for bispecific antibodies capable of recognizing the antigens used in both of the immunizations. However, some fraction of the B cells specific for the second antigen also have been involved in clonal expansion in response to the first antigen. These B cells are an obvious source for bispecific antibodies: with one of their rearranged light chain genes they can produce antibodies that have specificity for one of the two antigens, while with their other rearranged light chain gene they can produce antibodies specific for the other antigen. In both cases, individual B cells use one heavy chain protein to pair with both light chain proteins.

Hybridoma or other cloning technology is exploited to recover B cells with specificity for the second immunizing antigen. These B cells are then analyzed to determine whether they also carry a second rearranged heavy chain gene that confers specificity for the second antigen.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims that follow, unless the term “means” is used, none of the features or elements recited therein should be construed as means-plus-function limitations pursuant to 35 U.S.C. § 112, ¶6.

We claim:

1. A genetically modified animal with an engineered immunoglobulin heavy or light chain locus wherein a DNA cassette is inserted to select for productive V(D)J rearrangement, but prevents expression of functional immunoglobulin chain from the engineered immunoglobulin heavy or light chain locus.

2. The genetically modified animal of claim 1, wherein changes to one or more of the animal's immunoglobulin heavy or light chain genes allow for inducible inactivation and/or activation of expression of constant region-encoding parts of the genes.

3. An immunoglobulin light chain gene in the genetically modified animal of claim 1, wherein part or all of one or more constant region exons are placed in inverted reading frame orientation relative to rearranged VJ gene segments in the same immunoglobulin light chain gene.

4. An immunoglobulin protein expressed from the immunoglobulin gene of claim 3.

5. An immunoglobulin light chain gene in the genetically modified animal of claim 1, wherein a DNA cassette is inserted to prevent expression of constant region exons from rearranged VJ gene segments on the same chromosome.

6. An immunoglobulin protein expressed from the immunoglobulin gene of claim 5.

7. An immunoglobulin heavy chain gene in the animal of claim 1, wherein part or all of one or more constant region exons are placed in inverted reading frame orientation relative to rearranged V(D)J gene segments in the same immunoglobulin heavy chain gene.

8. An immunoglobulin protein expressed from the immunoglobulin gene of claim 7.

9. An immunoglobulin heavy chain gene in the animal of claim 1, wherein a DNA cassette is inserted to prevent expression of constant region exons from rearranged V(D)J gene segments on the same chromosome.

10. An immunoglobulin protein expressed from the immunoglobulin gene of claim 9.

11. Primary B cells, immortalized B cells or hybridomas expressing two or more functional variable heavy chain domains and/or two or more functional variable light chain domains derived from the genetically modified animal of claim 1.

12. Part of all of a heavy chain or light chain gene derived from the cells of claim 11.

13. The genetically modified animal of claim 1, that when immunized with one antigen and then with a second, different antigen generates B lymphocytes each capable of sequentially expressing or co-expressing two or more different antigen receptors and/or a bispecific antigen receptor.

14. The genetically modified animal of claim 13, wherein changes to one or more of the animal's immunoglobulin heavy or light chain genes allow for inducible inactivation and/or activation of expression of constant region-encoding parts of the genes.

15. An immunoglobulin light chain gene in the genetically modified animal of claim 13, wherein part or all of one or more constant region exons are placed in inverted reading frame orientation relative to rearranged VJ gene segments in the same immunoglobulin light chain gene.

16. An immunoglobulin protein expressed from an immunoglobulin gene of claim 15.

17. An immunoglobulin light chain gene in the genetically modified animal of claim 13, wherein a DNA cassette is inserted to prevent expression of constant region exons from rearranged VJ gene segments on the same chromosome.

18. An immunoglobulin protein expressed from an immunoglobulin gene of claim 17.

19. An immunoglobulin heavy chain gene in the animal of claim 13, wherein part or all of one or more constant region exons are placed in inverted reading frame orientation relative to rearranged V(D)J gene segments in the same immunoglobulin heavy chain gene.

20. An immunoglobulin protein expressed from an immunoglobulin gene of claim 19.

21. An immunoglobulin heavy chain gene in the animal of claim 13, wherein a DNA cassette is inserted to prevent expression of constant region exons from rearranged V(D)J gene segments on the same chromosome.

22. An immunoglobulin protein expressed from an immunoglobulin gene of claim 19.

23. Primary B cells, immortalized B cells or hybridomas expressing two or more functional variable heavy chain domains and/or two or more functional variable light chain domains derived from the genetically modified animal of claim 13.

24. Part or whole of a heavy or light chain derived from the cells of claim 23.

25. The genetically modified animal of claim 1, wherein two rearranged immunoglobulin heavy chain genes in individual B cells in the animal express gene products that do not homodimerize efficiently with each other.

26. B cells of the genetically modified animal of claim 25, wherein heterodimerization of the two gene products is enabled by a developmental or differentiation event, or can be induced.

27. Part or whole of a heavy or light chain derived from the cells of claim 26.

28. B cells of the genetically modified animal of claim 25, wherein homodimerization of the two gene products does not occur, or is disfavored relative to heterodimerization.

29. Part or all of a heavy chain or light chain gene derived from the cells of claim 28.

30. The genetically modified animal of claim 25, wherein changes to one or more of the animal's immunoglobulin heavy or light chain genes allow for inducible inactivation and/or activation of expression of constant region-encoding parts of the genes.

31. An immunoglobulin light chain gene in the genetically modified animal of claim 25, wherein part or all of one or more constant region exons are placed in inverted reading frame orientation relative to rearranged VJ gene segments in the same immunoglobulin light chain gene.

32. An immunoglobulin protein expressed from an immunoglobulin gene of claim 31.

33. An immunoglobulin light chain gene in the genetically modified animal of claim 25, wherein a DNA cassette is inserted to prevent expression of constant region exons from rearranged VJ gene segments on the same chromosome.

34. An immunoglobulin protein expressed from an immunoglobulin gene of claim 33.

35. An immunoglobulin heavy chain gene in the animal of claim 25, wherein part or all of one or more constant region exons are placed in inverted reading frame orientation relative to rearranged V(D)J gene segments in the same immunoglobulin heavy chain gene.

36. An immunoglobulin protein expressed from an immunoglobulin gene of claim 35.

37. An immunoglobulin heavy chain gene in the animal of claim 25, wherein a DNA cassette is inserted to prevent expression of constant region exons from rearranged V(D)J gene segments on the same chromosome.

38. An immunoglobulin protein expressed from an immunoglobulin gene of claim 37.

39. Primary B cells, immortalized B cells or hybridomas expressing two or more functional variable heavy chain domains and/or two or more functional variable light chain domains derived from the genetically modified animal of claim 25.

40. A heavy or light chain protein derived from the cells of claim 39.
